(19) United States
(12) Plant Patent Application Publication
(10) Pub. No.: US 2002/0144330 P1
(43) Pub. Date: Oct. 3, 2002

(54) Anthirrhinum plant named Balumwhit

(76) Inventor: Scott Trees, Shell Beach, CA (US)

Correspondence Address: ROCKEY, MILNAMOW & KATZ, LTD., Two Prudential Plaza, 180 North Stetson Avenue, Suite 4700, Chicago, IL, 60601, US

(21) Appl. No.: 09/808,202

(22) Filed: Mar. 14, 2001

(51) Int. Cl. A01H 5/00

(52) U.S. Cl. PLT/322

(57) ABSTRACT

A new and distinct Antirrhinum plant named Balumwhit characterized by its trailing habit and white flowers.

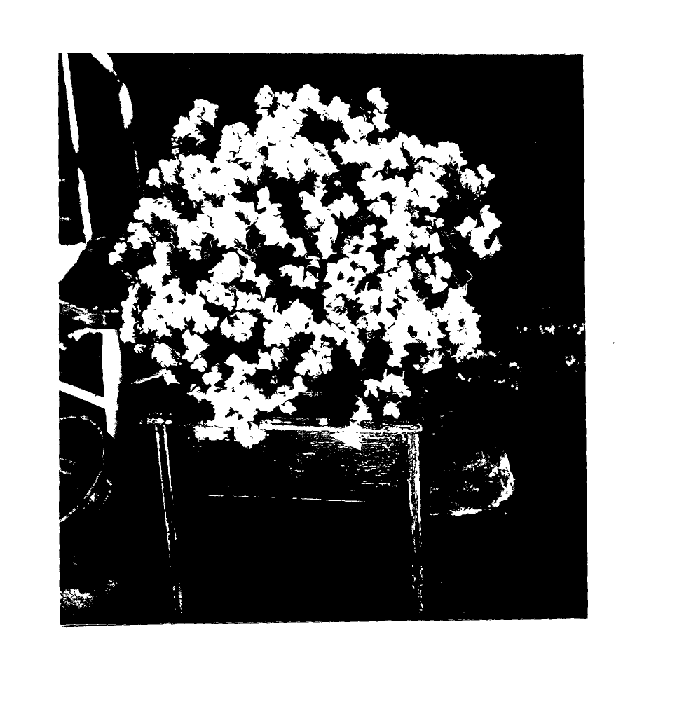

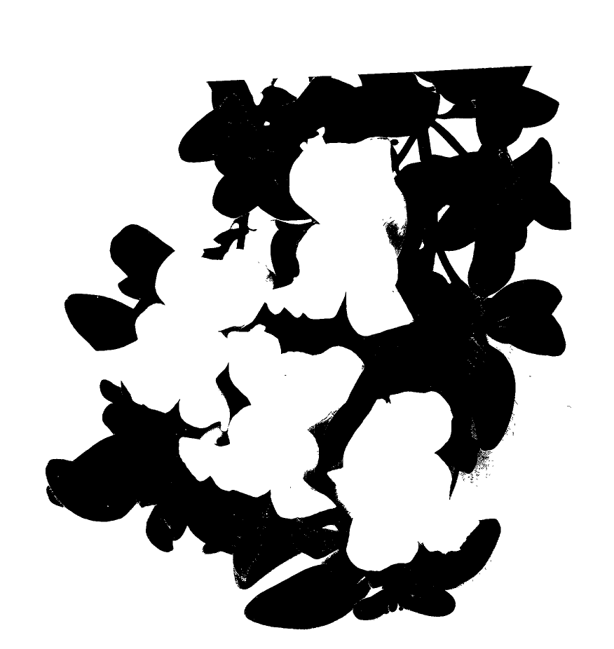

### BACKGROUND OF INVENTION

The present invention comprises a new and distinct Antirrhinum plant, hereinafter referred to by the cultivar name Balumwhit. This new cultivar was developed by the inventor through a controlled breeding program during August 1997 at Arroyo Grande, Calif. The objective of the breeding program was the development of Antirrhinum cultivars with trailing mounded habit, continuous flowering, excellent basal branching and small, dark green foliage.

The female parent of Balumwhit was the proprietary antirrhinum plant designated ‘white cut flower type’ which is characterized by its tall upright habit, white flowers and dark green foliage. The male parent of Balumwhit was the proprietary antirrhinum plant designated 116-D which is characterized by its vigorous habit and yellow flowers. The resulting seed was collected and germinated. From the flowering progeny, a plant was selected in April 1998 and initially designated 394-2.

Asexual reproduction of the new cultivar by terminal or stem cuttings taken during 1998 and 1999 at Arroyo Grande, Calif., U.S.A. has demonstrated that the characteristics of the new cultivar as herein described are firmly fixed and retained through successive generations of such asexual propagation.

### SUMMARY OF INVENTION

It was found that the cultivar of the present invention:

(a) exhibits a trailing habit,

(b) forms abundant white flowers and

(c) exhibits dark green foliage.

When the new cultivar of the present invention is compared to Chandelier Primrose Vein (United States Plant Patent Applied For) it is found that the new cultivar exhibits a somewhat less trailing habit, leaves of a different shape and color and flowers that differ in size and color as detailed in TABLE A.

### BRIEF DESCRIPTION OF PHOTOGRAPHS

The accompanying photographs show as nearly true as it is reasonably possible to make the same in color illustrations of this type, typical flower and foliage characteristics of the new cultivar.

The first photograph comprises a view of the growth habit of Balumwhit in a pot containing three plants.

The second photograph comprises a close-up view of typical flowers and leaves and is a good representation of actual flower color.

The plants were grown in a greenhouse at West Chicago, Ill., USA.

### DETAILED BOTANICAL DESCRIPTION

The Balumwhit cultivar has not been observed under all possible environmental conditions to date. Accordingly, it is possible that the phenotype may vary somewhat with variations in the environment, such as temperature, light intensity and day length.

The chart used in the identification of colors described herein is the R.H.S Colour Chart of The Royal Horticultural Society, London, England. The color values were determined on Sep. 26, 2000 in West Chicago, Ill. The readings were taken between 11:00 and 11:45 p.m. under natural light conditions. The plants were produced from cuttings taken from stock plants and grown for ten (10) weeks in a double poly carbonate covered greenhouse under conditions comparable to those used in commercial practice while utilizing a soilless growth medium and maintaining temperatures of approximately 72° F. during the day and approximately 65° F. during the night.

Classification:

Botanical.—Antirrhinum majus cultivar Balumwhit.

Commercial.—Trailing Snapdragon. Propagation:

Type cutting.—Terminal tip.

Time to Initiate Roots.—Approximately 10-14 days with the shorter times generally being experienced in the summer and the longer times in the winter.

Root Description.—Fibrous, branching.

Plant Description:

General Appearance and Form.—Mounded and trailing.

Plant length (from top of plant plane to lower flowers).—Approximately 18.3 cm.

Foliage width.—Approximately 30 cm.

Branching habit.—Freely branching. Pinching encourages branching.

Stem.—Diameter at lowest node approximately 2 mm, closest to 138B in color, densely covered

with short soft hairs.

Internode length.—Approximately 1 cm.

Foliage Description:

Form.—Single.

Shape.—Narrow elliptic, rounded to acute apex, attenuate base, entire margin.

Arrangement.—Opposite.

Venation pattern.—Arcuate.

Surface.—Upper and lower surface densely covered with short, soft hairs.

Color of mature foliage.—Upper surface closest to 137A with venation of 137C, lower surface closest to 137C with venation of 137C.

Size of mature leaves.—Approximately 2 cm in length, approximately 0.8 cm in width.

Petiole.—Length is approximately 4 mm, color is 137C.

Flower Description:

Flowering habit.—Balumwhit is freely flowering under outdoor growing conditions with substantially continuous blooming from spring until fall.

Flower arrangement.—Terminal racemes.

Flower.—Approximately 3.3 cm in length, bilabiate. Lip texture is smooth, margin is entire and somewhat undulating. Upper lip is 2 parted, approximately 1.9 cm in length, approximately 1.7 mm in width, lighter than 150D with very fine venation of 64C. Lower lip is 3 parted, approximately 1.2 cm in length, approximately 1.8 cm in width, lighter than 150D with 2 areas of 5A where lip meets palate . The palate is 155D with hairs of 155D. Inside of throat is lighter than 150D with hairs of 3C, outside of throat is lighter than 150D.

Corolla tube.—Approximately 2.7 cm in length and 0.8 cm in width.

Peduncle.—Length is approximately 9 mm, diameter is approximately 1 mm, color is 138B and surface is covered with short soft hairs.

Flower bud.—Length is Approximately 2 cm, width is approximately 1 cm, color is between 145B and 145C texture is pubescent.

Calyx.—5 sepals, approximately 7 mm in length, 4 mm in width, ovate with acute apex covered with long soft hairs, color is between 138A and 138B.

Reproductive Organs.—Androecium: 4 stamens, 2 are 1.5 cm in length, 2 are 1 .1cm in length, closest to 155D in color. Anthers approximately 3 mm in length, 16A. Pollen color is closest to 14C. Gynoecium: One pistil. Stigma length is 0.5mm, color is 144B. Style length is approximately 1.2 cm long, color is 150D. Ovary is approximately 2 mm in diameter, 150D. hairy.

Seed productuin: Seed production has not been observed.

Disease resistance: Resistance to pathogens is not claimed.

I claim:

1. A new and distinct cultivar of Antirrhinum named ‘Balumwhit’ substantially as herein shown and described, which:
(a) exhibits a trailing growth habit,
(b) forms abundant white flowers and
(c) exhibits dark green foliage.
TABLE A

| CHARACTERISTIC | BALUMWHIT | PRIMROSE |
|---|---|---|
| PLANT LENGTH (FROM | 18.3 CM | 29.3 CM |
| TOP OF PLANT PLANE | | |
| TO BOTTOM OF | | |
| FLOWERS)-CM | | |
| MATURE LEAF | 2 × .8 | 1.2 × 1 |
| LENGTH × WIDTH-CM | | |
| LEAF SHAPE | NARROW ELLIPTIC | OVAL |
| COLOR OF UPPER | 137A | 191A |
| SURFACE OF | | |
| MATURE FOLIAGE | | |
| COLOR OF UPPER | LIGHTER THAN | 155D WITH |
| LIP OF FLOWER | 155D WITH | VENATION |
| FLOWER | VENATION OF 72D | OF 70A |
| LENGTH × WIDTH-CM | 2.3 × 3.3 | 1.3 × 3.1 |
